SEQUENCE LISTING (32 sequences)

SEQ ID NO: 1 - DNA, 23 bp, Homo sapiens
gcctgtttgaggacctgtcgtta

SEQ ID NO: 2 - DNA, 23 bp, Homo sapiens
tggttatagagcagttaaggcac

SEQ ID NO: 3 - DNA, 24 bp, Homo sapiens
ggcagattcaataggataaataga

SEQ ID NO: 4 - DNA, 24 bp, Homo sapiens
gtaacccctcaggttctcaagtct

SEQ ID NO: 5 - DNA, 24 bp, Homo sapiens
ataactgggctaggaatggaaata

SEQ ID NO: 6 - DNA, 22 bp, Homo sapiens
gacttcccaattcaggaggact

SEQ ID NO: 7 - DNA, 24 bp, Homo sapiens
gcattctgggcaacaaagtgaaac

SEQ ID NO: 8 - DNA, 24 bp, Homo sapiens
gagataacccaggcaagaacagga

SEQ ID NO: 9 - DNA, 24 bp, Homo sapiens
tgagtcaggagaatcacttggaac

SEQ ID NO: 10 - DNA, 24 bp, Homo sapiens
ctcttccatgaagtagctaagcag

SEQ ID NO: 11 - DNA, 24 bp, Homo sapiens
ggacttaccacaggcaatgtgact

SEQ ID NO: 12 - DNA, 24 bp, Homo sapiens
tagacttcactattcccatctgag

SEQ ID NO: 13 - DNA, 24 bp, Homo sapiens
cagcaaacttcatgtgacaaaagc

SEQ ID NO: 14 - DNA, 24 bp, Homo sapiens
caatggtaacctactatttacgtc

SEQ ID NO: 15 - DNA, 24 bp, Homo sapiens
tttctcagtctccataaatatgtg

SEQ ID NO: 16 - DNA, 24 bp, Homo sapiens
gatgttgtattagtcaatgttctc

SEQ ID NO: 17 - DNA, 24 bp, Homo sapiens
ctatcccactgtattattcagggc

SEQ ID NO: 18 - DNA, 24 bp, Homo sapiens
tgagtctccaggttgcaggtgaca

SEQ ID NO: 19 - DNA, 25 bp, Homo sapiens
gtagatacatacatatgatgaatgc

SEQ ID NO: 20 - DNA, 23 bp, Homo sapiens
tattaatgtgtgtccttccaggc

SEQ ID NO: 21 - DNA, 25 bp, Homo sapiens
gcaaatttgtggatgggattaacag

SEQ ID NO: 22 - DNA, 24 bp, Homo sapiens
aagctaaatgtctgtagttattct

SEQ ID NO: 23 - DNA, 24 bp, Homo sapiens
ccctgggctctgtaaagaatagtg

SEQ ID NO: 24 - DNA, 24 bp, Homo sapiens
atcagagcttaaactgggaagctg

SEQ ID NO: 25 - DNA, 20 bp, Homo sapiens
gaaatgtgctttgacaggaa

SEQ ID NO: 26 - DNA, 22 bp, Homo sapiens
caaaaagggacatatgctactt

SEQ ID NO: 27 - DNA, 22 bp, Homo sapiens
ccatctctgtctccatctttgt

SEQ ID NO: 28 - DNA, 22 bp, Homo sapiens
cccctgtctatggtctcgattc

SEQ ID NO: 29 - DNA, 20 bp, Homo sapiens
taatgagagttggaaagaaa

SEQ ID NO: 30 - DNA, 20 bp, Homo sapiens
cccattgttgctacttgaga

SEQ ID NO: 31 - DNA, 20 bp, Homo sapiens
tgaaccttcctagctcagga

SEQ ID NO: 32 - DNA, 20 bp, Homo sapiens
tctggagaatccaattttgc

(19) United States
(12) Patent Application Publication
(10) Pub. No.: US 2006/0263785 A1
(43) Pub. Date: Nov. 23, 2006

(54) Detection of aneuploidy

(76) Inventor: Roger Quaife, Singapore (SG)

Correspondence Address: CHERSKOV & FLAYNIK;The Civic Opera Building, Suite 1447, 20 North Wacker Drive, Chicago, IL, 60606, US

(21) Appl. No.: 10/874,958

(22) Filed: Jun. 22, 2004

(30) Foreign Application Priority Data
Jun. 27, 2003 (SG) 200303715-7

(51) Int. Cl. C12Q 1/68; C07H 21/04; C12P 19/34; C12M 1/34

(52) U.S. Cl. 435/6; 435/287.2; 435/91.2; 536/24.3

(57) ABSTRACT

A method for detecting aneuploidy of a chromosome comprises: a) obtaining a biological sample from a patient; b) preparing DNA from said sample for subsequent analysis; c) subjecting the DNA of said sample to a multiplex PCR reaction, using a multiplicity of markers, at least two per said chromosome of which have a heterozygosity frequency sufficiently high to minimise the possibility that, if two alleles of that chromosome would normally be present in a normal diploid individual, those two alleles would both comprise the same variant of said STR (i.e. would be homozygous); d) comparing the results of said multiplex reaction with the results which might be expected from a normal diploid individual; and e) thereby assessing the probability that said patient might be suffering from chromosomal aneuploidy. Accordingly, a diagnostic kit for use in DNA analysis for detecting aneuploidy of a chromosome comprises: a) highly conserved STR markers which have a heterozygosity frequency with respect to said chromosome which is sufficiently high to minimise the possibility that, if two alleles of that chromosome would normally be present in a diploid individual, those two alleles would both comprise the same variant of said STR marker (i.e. be homozygous); and b) other standard reagents for use in multiplex PCR.

### TECHNICAL FIELD

The present invention relates to a diagnostic test that can be used for detection of aneuploidies. In particular, the present test can be used for prenatal screening.

### BACKGROUND TO THE INVENTION

In cytogenetics, prenatal diagnosis for chromosome abnormality is routinely undertaken by full karyotype analysis of chromosomes from cultured cells. This is regarded as the “gold standard” for testing, and involves culturing of the cells (from 15-20 ml of amniotic fluid, chorionic villi, skin or products of conception), harvesting of the dividing cells, making of slides, staining after enzyme digestion, and skilled analysis of the resulting enzyme banded and stained chromosomes. It is both time consuming and labour intensive, Resolution of this test is up to 3-4 Mb, depending on die quality of the initial preparation.

Alternatively, interphase-fluorescent in-situ hybridisation (FISH) is employed in testing for the copy number of chromosomes 13, 18, 21 and the sex chromosomes, The results of FISH can be obtained within 24-28 hours. It allows the visualization of specific nucleic acid sequences within a cellular preparation in which cells from amniotic fluid samples have been treated using cytogenetic protocols and then attached to glass slides. Specifically, FISH involves the precise annealing of a single stranded, fluorescently labelled DNA probe to complementary target sequences. The hybridisation of the probe with the cellular DNA site is visible by direct detection using a fluorescence microscope. It too is time consuming, labour intensive and expensive, Quantitative Fluorescent PCR (QF-PCR; sometimes referred to as Amnio-PCR) is a new method employing specific DNA oligonucleotides (labelled with fluorescent tags) for the amplification of target DNA sequences. All target sequences are amplified concurrently in a single reaction (ie multiplex). These amplified DNA fragments are electrophoresed on a capillary sequencer in which a laser detects the fragments as quantitative peaks.

The oligonucleotides (primers) used for amplification in QP-PCR are short tandem repeat (STR) DNA sequences. The amplified DNA sequences are tetranucleotide repeats which are highly polymorphic and conserved (Adinolfi et al, 1995). As such, they act as markers for a particular chromosome within an homologous pair and tend to be suitable when the primers are multiplexed (Utah Marker Development Group, 1995), If these primers are also labelled with a fluorescent tag, the amplified products can be detected as quantitative peaks using an automated capillary DNA sequencer. A smaller quantity of sample is required (eg 2 ml of amniotic fluid, or a single frond of chorionic villus or product of conception). Resolution of this test can be as high as a single base difference, due to the sensitivity of the sequencer. In addition to screening for aneuploidies, QF-PCR can also be adapted to allow concurrent single gene and chromosome analysis or separately for a wide range of infectious disease investigations.

Normal individuals with polymorphic markers will have two peaks for chromosomes 21, 13 and 18 (normal diallelic, ratio 1:1). Alternatively, trisomic polymorphic individuals will either have three equal sized peaks (trisomic triallelic, ratio 1:1:1) or two uneven sized peaks, the larger peak representing two chromosomes and the smaller a single chromosome (trisomic diallelic, ratio 2:1). The situation for the X and Y chromosomes is similar but, in monosomy X females (Turner's Syndrome), there will be a single peak if the marker used is normally diallelic (ratio 1:1) for the X alone. However, if the marker employed recognizes sequences on both the X and Y chromosomes, it could be dialletic for the X in normal females but, in males, there would be a single peak for both the X and Y chromosomes.

Since STR's are highly polymorphic, virtually all individuals will have at least one heterozygous marker. Nonetheless, for confirmation of the result, at least two markers are required to be concurrently heterozygous. In recent studies, three or four markers have been employed per chromosome. However, the markers used in the present invention have been adjusted, based upon the heterozygosity frequency of each and calculations on the probability of any two or more therefore being concurrently informative.

Furthermore, in the past, recognition of X and Y numerical disorders using QF-PCR has also been hampered by the low polymorphism information content (PIC) of the markers employed. Recently, alternative markers have been introduced which tend to overcome this deficiency (Cirigliano et al, 2001), In the method of the present invention, two of these alternative markers, one of which has been modified, were included in a single multiplex reaction, along with several markers for the autosomal chromosomes 13, 18 and 21.

### SUMMARY OF THE INVENTION

Accordingly, the present invention provides a method for detecting aneuploidy of a chromosome comprising:
a) obtaining a biological sample from a patient;
b) preparing DNA from said sample for subsequent analysis;
c) subjecting the DNA of said sample to a multiplex PCR reaction, using a multiplicity of markers, at least two per said chromosome of which have a heterozygosity frequency sufficiently high to minimise the possibility that, if two alleles of that chromosome would normally be present in a diploid individual, those two alleles would both comprise the same variant of said STR (i.e. would be homozygous);
d) comparing the result of said multiplex reaction with the results which might be expected from a normal diploid individual; and
e) thereby assessing the probability that said patient might be suffering from chromosomal aneuploidy.

The markers used in the present invention, being highly polymorphic and conserved, are likely to be present, in different forms, on each of a pair of chromosomes. The results of testing based on these markers may indicate if aneuploidy is present. For the trisomic case, there may be three equal-sized peaks (three different variants of the marker) or two uneven-sized peaks (the same variant present on two chromosomes, with a different one on the third chromosome). If the results of the test indicate a possible positive for aneuploidy, more specific tests can be undertaken to confirm the results (for example, cytogenetic analysis).

The testing regime of the present invention is particularly suitable for large-scale screening of patients, for prenatal detection of aneuploidy. It is sufficiently accurate, yet simple and cost-effective to administer, to warrant widespread use as a standard prenatal test.

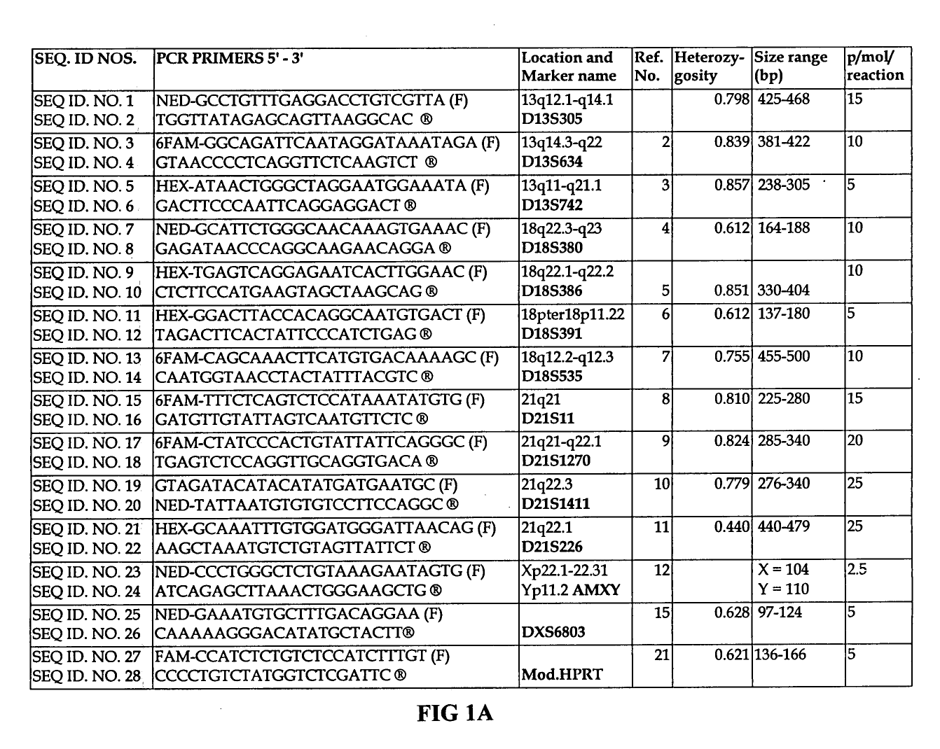

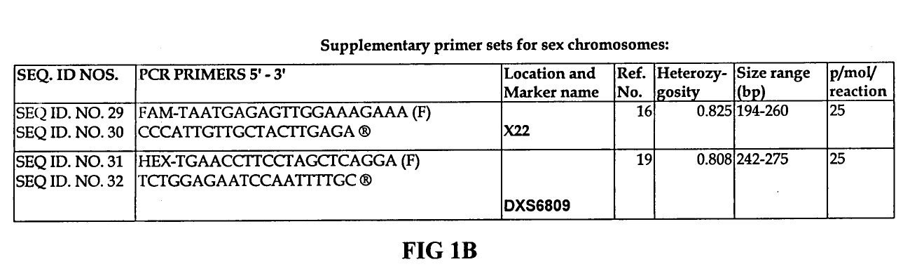

A diagnostic kit for use in the testing method of the present invention will, for example, comprise the fourteen (14) pairs of primers shown in FIG. 1. These amplifying polymorphic markers comprise markers for both sex chromosomes and autosomes. Suitable concentrations for the reaction are indicated in the third column of FIG. 1.

One of these markers (HPRT), present in the Hypoxanthine Guanine Phosphoribosyltransferase gene, has been modified by assigning new hybridisation sites. These sites have not previously been used. The new primer is shown in FIG. 1, and is now referred to as “Mod.HPRT”.

Heterozygosity probabilities based upon observed polymorphic frequencies within a largely Asian population sample have been calculated. These polymorphic frequencies have not previously been derived.

Accordingly, the diagnostic kit of the present invention, for use in DNA analysis for detecting aneuploidy of a chromosome, will comprise:
a) highly conserved STR markers which have a heterozygosity frequency with respect to said chromosome which is sufficiently high to minimise the possibility that, if two alleles of that chromosome would normally be present in a diploid individual, those two alleles would both comprise the same variant of said STR marker (i.e. be homozygous); and
b) other standard reagents for use in multiplex PCR.

Quantitative Fluorescent Polymerase Chain Reaction (QF-PCR) is a rapid and efficient method for the detection of common aneuploidies (ie abnormality in chromosome copy number). QF-PCR involves 3 stages, being DNA extraction, PCR amplification of DNA sequences, and fragment analysis using a capillary DNA sequencer.

Indications for utilising the QF-PCR method of the present invention would in general be the same as in routine cytogenetics and, for some of these indications, it may be considered as a stand-alone test. These nay include the serum triple/double test result; advanced maternal age; and choroid plexus cysts, all of which are more often than not associated with aneuploidy. Maternal anxiety, particularly if there is a family history of aneuploidy, may also be a reason for conducting prenatal testing. In such cases, QF-PCR may be an appropriate, albeit slightly less comprehensive, alternative to cytogenetic analysis, since it allows the rapid, high throughput detection of 95% of chromosome abnormalities (Cuckle and Wald, 1990) but with the parsimonious use of materials, labour and time. Both the PCR amplification and DNA based sequencer detection are fully automated. This enables prenatal diagnosis where the skill levels and high consumable costs required for conventional cytogenetic analysis, or FISH, are not readily available. Furthermore, the test has a rapid turn-around time, and automation means that the labour intensity required by conventional cytogenetic methods is much reduced. Therefore, QF-PCR is capable of processing relatively large numbers of samples concurrently. One or two scientists can extract DNA, amplify and analyse more than forty samples, all within 24-48 hours, thereby eliminating the need for arbitrary limits such as maternal age, anti allowing more patents access to prenatal diagnosis for chromosome aneuploidy. With the routine use of real-time ultrasound, the risks associated with die amniocentesis procedure have declined. Furthermore, the fluid volume required for QF-PCR is relatively small. However, intrauterine lavage may be an alternative, minimally invasive method of obtaining foetal cells and this is already proving successful (Bussani et al, 2002; Bulmer et al, 2003). It is therefore not unreasonable to suggest that all pregnancies can be screened for aneuploidies and common single gene abnormalities using QF-PCR. The health care savings both to parents and the state would easily support such an initiative, The cost per test using this methodology should not be greatly in excess of the present triple test but, as an alternative, it is almost 100% accurate.

### DETAILED DESCRIPTION OF THE INVENTION

### Example 1

Materials and Methods

Samples

A total of 1115 samples were investigated. These were split into two separate batches: the first (a) consisted of 500 and the second (b) consisted of 615 samples. In all cases, DNA was routinely extracted from native amniotic fluid samples (1.0-2.0 ml) or long-term cultures and chorionic villus samples (CVS) or the products of conception (POC), again either native or cultured. The methodology was as described by the manufacturer (Bio-Rad), using their InstaGene Matrix, except 100 μL was used with amniotic fluid and all culture types and 200 μL for CVS and POC, either with a prior wash with phosphate buffered saline (pH 7.4) for clear amniotic fluid and cultures, or with water for either blood-stained amniotic fluid or uncultured CVS or POC. Sample batch (a) consisted entirely of amniotic fluid samples and the gestation varied between 14-28 week.

PCR

The amplification cycle for all samples was consistent and followed the protocol described by Mann et al (2001): an initial denaturation at 94° C. for 3 minutes, followed by 25 cycles at 94° C. for 30 seconds, annealing at 57° C. for 1 minute and extension at 71° C. for 2 minutes. There followed a final extension step at 71° C. for 5 minutes and incubation at 60° C. for 1 hour. The amplification products were then held at 4° C. until required.

The primers used differed between the trials. Initially (apart from changes in concentrations) the multiplex primer combination followed that of Mann et al (2001), but after the results of the blind trial from batch (a) were available, this was changed, The reason was both because of marker heterozygosity frequencies and the need for the ability to recognise Turner's syndrome and triple X females within a single multiplex. For ease of application and cost, it was felt inappropriate to have separate multiplexes for autosomal and sex aneuploidy (below). Primer concentrations were again adjusted accordingly.

Master mixes containing primers, but excluding Taq polymerase (Promega) and template DNA, were made in advance, aliquoted in 14.5 μL, lots and stored at −20° C. until required, when they were returned to room temperature. At this point, the template (10 μL) and Taq polymerase (2U) were added, thereby returning the mix to the required 25 μL, reaction volume ×1 concentration. Amplification was carried out using a Biometra T1 thermocycler.

Product Analysis

Amplification products were run using an ABI 3100 four-channel capillary DNA sequencer with 3100 Avant data collection software version 1.0; GeneScan version 3.7.1 for raw data fragment analysis (based on the internal size standard) and Genotype version 3.7 for allele assignment and labeling (within the expected size ranges). Dye set DS 30 with GeneScan-500 Rox was the internal lane standard (Applied Biosytems). The sample capillary injection time was set at either 10 or 15 seconds, and 3 or 1 Kv respectively. Each amplified sample (2 μL) was added to 9.5 μL of formamide and 0.5 μL of size standard, in a MicroAmp (Applied Biosystems) optical 96 well reaction plate. Prior to electrophoresis, this mix was denatured for 2 minutes at 95° C. and cooled for 3 minutes at −20° C.

At analysis, the peak scale (in arbitrary fluorescence units) was varied from 1,000 to 10,000, depending upon the level of amplification in each sample. Low levels of amplification seemed mainly due to early gestation (<16 weeks), and hence the number of cells originally available to produce template DNA (results below).

Method

2 ml of uncultured sample was treated with a commercial reagent for DNA extraction following the protocol provided by the manufacturer. This DNA was then amplified by PCR, employing 3 or 4 primer pairs per chromosome (chromosomes 13, 18, 21 and sex chromosomes). All the markers were labelled with fluorescent tags. The amplified products were then electrophoresed using a capillary DNA sequencer in which a laser detected individual fragments based on the fluorescence emitted by the fluorescent tags. This fluorescence was quantified as peaks. Normal individuals with polymorphic markers will have 2 peaks for chromosome 13, 18 and 21 (ie normal diallelic with peak ratio 1:1, indicating presence of 2 alleles). Alternatively, trisomic polymorphic individuals will either have 3 equal sized peaks (trisomic triallelic, peak ratio 1:1:1, presence of 3 alleles) or 2 uneven sized peaks, the larger peak representing 2 chromosomes and the smaller a single chromosome (trisomic diallelic, ratio 2:1, presence of 2 alleles). Since the STR's used are highly polymorphic, almost all individuals will have at least 1 heterozygous marker. A minimum of 2 informative markers is required for confident interpretation. A single peak is described as uninformative and may indicate either allele homozygosity or a monosomic affected individual.

Results

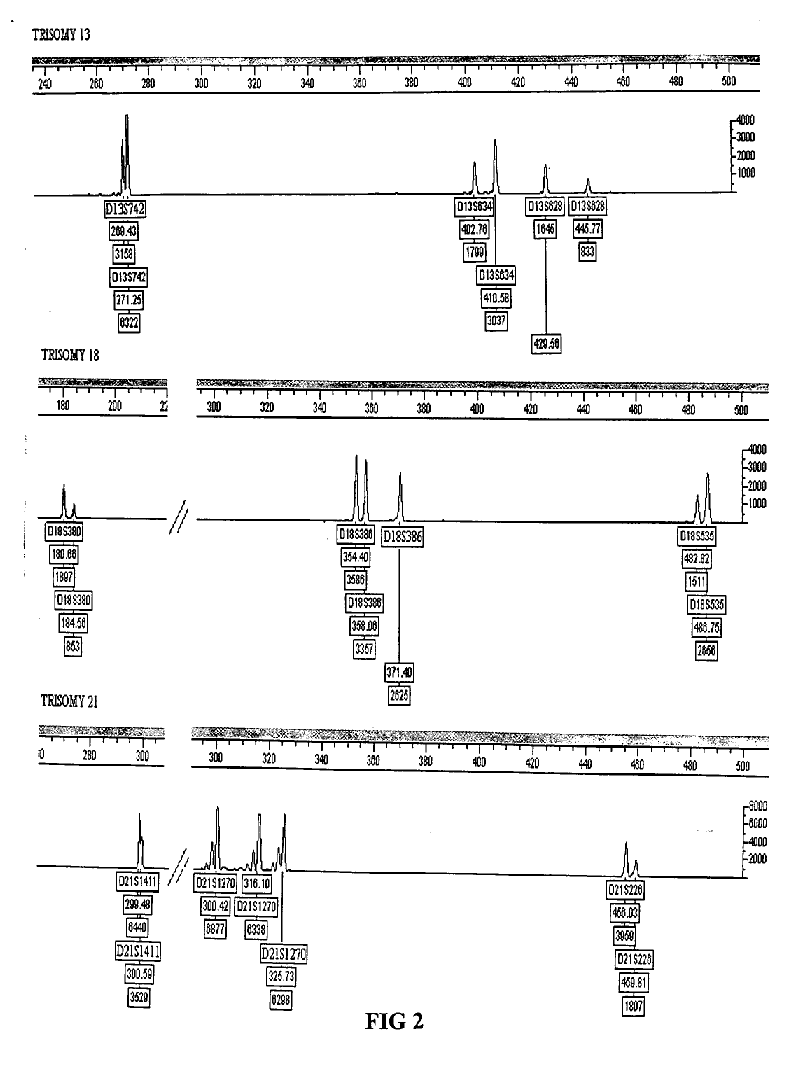

Initially, peak ratios between 0.8 and 1.4 were assigned as normal, and peak ratios greater than 1.8 or less than 0.65 were assigned as abnormal (Adinolphi et al, 1997; Mann et al, 2001). Subsequently, these were adjusted to assign peak ratios of less than 0.69 or greater than 1.85 as abnormal, Normality ranged from 0.69 to 1.85. However, an observed overlap at 1.7 between normal and abnormal occurred in some of the markers used. Alternatively, three peaks of equal height would also represent abnormality. Normally, a minimum of two polymorphic markers would be required to confidently assign either normality or abnormality. Sample batch (a) detected a total of 9 abnormal results representing 1.8% of the total, and batch (b) detected 34 (5.5% of that sample) (Table 1; FIG. 2). The results for (a) exactly matched those previously seen by conventional cytogenetic analysis. However, batch (b) was discordant in two POC samples (see below).

TABLE 1
Chromosome abnormalities detected using QF-PCR.

| | AF batch | AF batch | POC batch | CVS batch |
|---|---|---|---|---|
| Abnormality | (a) | (b) | (b) | (b) |
| Trisomy 13 | 2 | 1 | 3 | |
| Trisomy 18 | 3 | 1 | 1 | 1 |
| Trisomy 21 | 1 | 4 | 6 | |
| Trisomy 18 mosaic | | 1 | | |
| Triploid XXX | | 1 | 1 | |
| Triploid XXY | | | 3 | |
| XYY | 2 | | | |
| Klinefelter | 1 | 3 | 1 | |
| Turner | | | 3 | |
| Triple X | | 2 | 1 | |
| Tetraploid XXYY | | | Missed | |
| Total* | 9 | 13 | 19 | 1 |

*Plus one assumed complete uniparental disomic POC not detected cytogenetically.

After the first batch of results became available, the probability of two or more markers being heterozygous for chromosomes 21, 13 and 18 were calculated, based upon the observed heterozygosity rate for each marker within the sample (Table 2;FIG. 1).

TABLE 2
Allele size range (in bp) and heterozygosity, sample batches (a) and (b) combined.

| | | | | | Alleles: | Allele: most |
|---|---|---|---|---|---|---|
| Chromosome | Marker | Label | Size | Heterozygosity | number | common |
| D13 | S628 | NED | 429-465 | 0.652 | 11 | 429 |
| | S305 | NED | 425-468 | 0.798 | 32 | 450 |
| | S634 | 6-FAM | 381-422 | 0.839 | 31 | 397 |
| | S742 | HEX | 238-305 | 0.857 | 45 | 269 |
| D18 | S380 | NED | 164-188 | 0.612 | 12 | 180 |
| | S386 | HEX | 330-404 | 0.851 | 51 | 354 |
| | S391 | HEX | 137-180 | 0.612 | 15 | 159 |
| | S535 | 6-FAM | 455-500 | 0.755 | 21 | 486 |
| D21 | S11 | 6-FAM | 225-280 | 0.810 | 32 | 246 |
| | S1270 | 6-FAM | 285-340 | 0.824 | 41 | 299 |
| | S1411 | NED | 276-340 | 0.779 | 30 | 307 |
| | S226 | HEX | 440-479 | 0.440 | 24 | 459 |
| X | S6803 | HEX | 97-124 | 0.628 | 22 | 116 |
| | Mod.HPRT | 6-FAM | 136-166 | 0.621 | 15 | 149 |
| | AMEL | NED | 104 | N/A | N/A | N/A |
| Y | AMEL | NED | 110 | N/A | N/A | N/A |

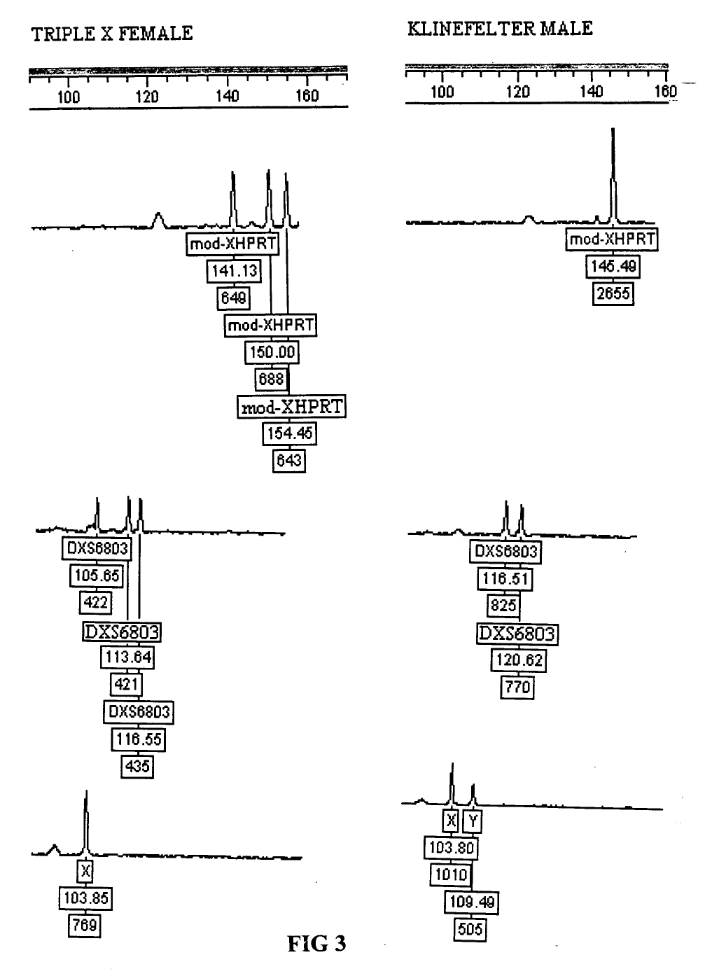

Thus, the probabilities were: for chromosome 21 P=0.9431, for chromosome 18=0.9341 and for chromosome 13=0.9098. The lower probability derived for chromosome 13 was due to the three markers (as opposed to four, with chromosome 21 and 18) routinely used for this chromosome and the fact that one of these three markers (D13S628) had a relatively low heterozygosity rate of 0.652. For these reasons, sample (b) was analyzed using an alternative marker combination where D13S305 (which has a heterozygosity frequency of 0.798 in the relevant population sample) replaced D13S628. In addition, two polymorphic markers for the X chromosome were introduced. These were DXS6803 and a modified form of HPRT. The latter has a new primer pair amplifying a fragment in the range 136-166 bp. This allowed it to be multiplexed and for the resulting fragment to be situated at an available site using FAM. In addition, two other polymorphic X markers were employed. These were X22 (heterozygosity frequency 0.825) situated on the pseudoautosomal region PAR2(Xq/Yq) and DXS6809 (heterozygosity frequency 0.808). Both were used in a separate mix if Mod.HPRT and DXS6803 proved uninformative (thereby allowing the possibility of identifying Turner's syndrome). This occurred in approximately 7% of cases in batch (b) (44 samples). In all but two of these samples, the use of these additional markers removed this ambiguity and allowed the identification of Turner's syndrome in 3 cases, confirmed by reference to the previous cytogenetic result. The remaining two samples proved, by cytogenetic analysis, to be normal female. Thus, according to the present results, the probability of these markers being false positive for Turner's is 1 in 307. However, in all apparently uninformative cases, it is recommended that either FISH be applied, or that a full cytogenetic analysis be awaited. The use of these four polymorphic X and X/Y markers and the amelogenin marker also allows the diagnosis of triple X females, as well as XYY and XXY males (FIG. 3).

For all the markers used, the allele size range appeared to be either less than, or different from, those previously reported. On average, the range for samples (a) and (b) was 45 bp between the smallest and largest allele, whereas in other population samples (Mann et al, 2001, Bili et al, 2002) it was 55 bp (Tables 2 (above) and 3). The average number of alleles seen was 27.

TABLE 3
Sample failure, contamination and marker heterozygosity.

| | SAMPLE - BATCH: | | | |
|---|---|---|---|---|
| | (A) | | (B) | |
| Failures (repeated ×2) | 7 AF | (1.4%) | 21 AF | (3.4%) |
| Maternal contamination | 3 AF | (0.6%) | 9 AF | (1.5%); |
| | | | 3 POC | (0.5%) |
| Single informative marker: | | | | |
| 13 | 33 AF | (6.6%) | 23 AF | (3.7%); |
| | | | 1 POC | (0.1%) |
| 18 | 26 AF | (5.2%) | 23 AF | (3.7%); |
| | | | 1 POC | (0.1%) |
| 21 | 21 AF | (4.2%) | 20 AF | (3.2%); |
| | | | 1 POC | (0.1%); |
| | | | 2 CVS | (0.3%) |
| Uninformative: | | | | |
| 13 | 3 AF | (0.6%) | 1 AF | (0.2%) |
| 18 | 3 AF | (0.6%) | 2 AF | (0.3%) |
| 21 | 6 AF | (1.2%) | 1 POC | (0.2%) |

| | | | |
|---|---|---|---|
| X | N/A | 2 POC | (0.3%) |

A total of 7 samples in batch (a) and 21 in (b) failed to amplify. This was despite repeating both the amplification on the original DNA extraction, and doing a repeat extraction. This represents a 2.5% failure rate. However, the majority of these failures was below 16 weeks gestation and noted to have a sparse number of cells prior to cytogenetic culturing, None of the other samples failed to amplify and all gave satisfactory results. Since the method of DNA extraction used did not remove proteins, the 260/280 nanometer (nm) ration was below 1.8 and, as such, the optical density at 260 nm would be unreliable in calculating the DNA concentrations. It was thus not possible to prove that the failed samples had insufficient DNA for amplification, but this can be implied.

Staining by maternal blood was noted prior to DNA extraction in 3 samples in batch (a) and 12 in (b), They all amplified successfully but subsequently produced marker profiles that were unusual compared to normal. All the markers produced this characteristic pattern and this allows the recognition of such contamination.

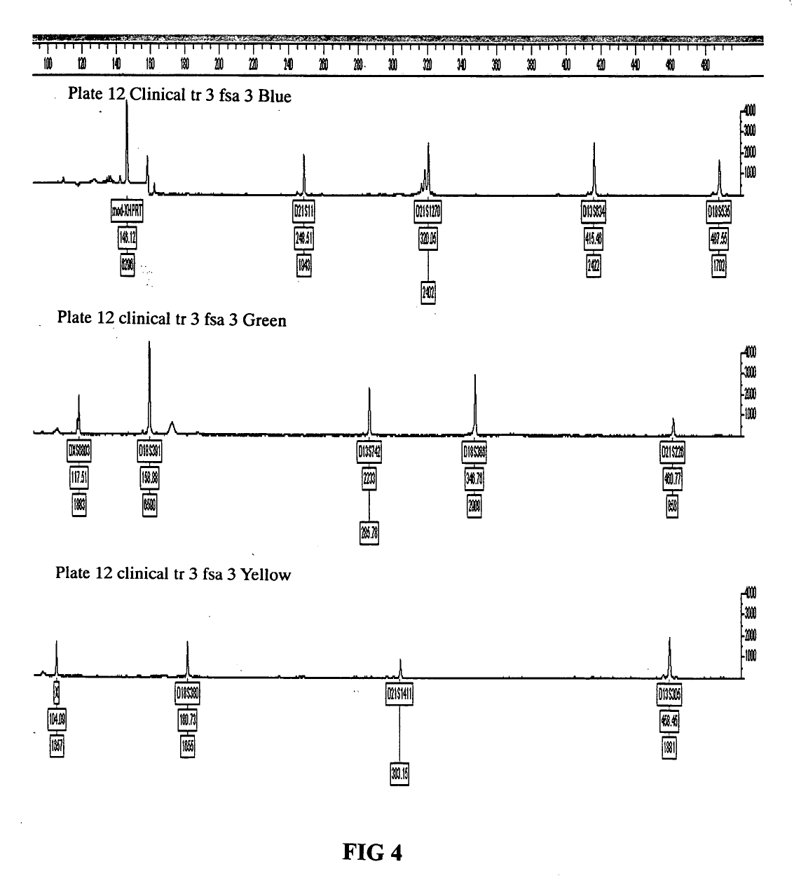

Three samples were of special note. The first (a POC) produced a profile in which each marker was apparently homozygous (FIG. 4). A repeat analysis gave the same result. The probability of this occurring by chance alone is approximately 1 in 43-44 million. An alternative explanation might be that this foetus was uniparentally disomic for every chromosome, perhaps arising from the fertilization of a nullisomic ovum with subsequent replication into the diploid state. The previous cytogenetic result was a normal female, which would be expected in such a case.

Another POC was found to be tetraploid by cytogenetic analysis, but produced a normal male profile using QF-PCR. Here again it may be that endoreduplication had produced the tetraploid state but, in this case, would not be recognized by QF-PCR since each homologue is simply duplicated.

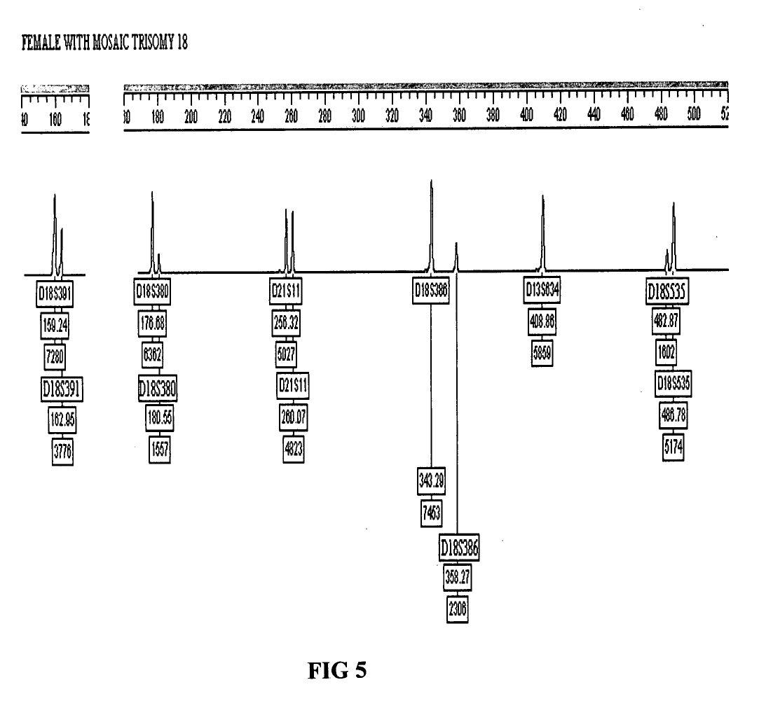

Finally, one sample, being an AF (amniotic fluid) sample, was recognized as a possible mosaic for Trisomy 18 (FIG. 5) and this was confirmed by reference to the previous cytogenetic analysis where 40% of cells seen were trisomic. The profile appeared similar to that seen in maternal contamination, but limited to chromosome 18, all the other markers being normal, Furthermore, examination of the four marker peak areas revealed either a 4:1 (D18S380) or a 3:2 (D18S386; D18S535) or a 2:1 (D18S391) ratio, presumably depending, on the mixture (or not) of alleles from normal and trisomic cells. Thus, a trisomic diallelic together with a monoallelic normal could present as either a 4:1 or a 3:2 ratio. A trisomic diallelic with a normal diallelic would be a 3:2 ratio. A 2:1 ratio can only represent pure trisomy.

Discussion

These results suggest that QF-PCR is a robust method for the prenatal detection of major chromosome abnormality. Apart from 2 specimens, all the abnormalities present in the samples were detected and there was no discordance between the results obtained with QF-PCR and conventional cytogenetics,

In addition to the detection of chromosome abnormality, such PCR based methodology can also be adapted to allow single gene analysis to be performed concurrently. In populations where a gene is segregating at levels highly in excess of the mutation rate, the most common mutation can be detected. For example, this can be achieved by means of a fluorescently labeled primer designed for use with the Amplification Refractory Mutation System (ARMS).

### REFERENCES

Adinolfi, M. C., Sherlock, J., Pertl, B. (1995)

Rapid detection of selected aneuploidies by quantitative fluorescent PCR. BioEssays, 17(7),661-664

Utah Marker Development Group (1995)

A collection of ordered tetranucleotide repeat markers from the human genome. Am. J. Hum. Genet., 57,619-628

Cirigliano, V., Lewin, P., Szzpiro-Tapies, S., Fuster, C., Adinolfi, M, (2001)

Assessment of new markers for the rapid detection of aneuploidies by quantitative fluorescent PCR (QF-PCR). Ann. Hum. Genet. 65,421-427

Cuckle, H. S., Wald, N.J. (1990)

Screening for Down's Syndrome. In: Lilford, R. (ed.) Prenatal Diagnosis and Prognosis, Vol. 11 London: Butterworths, 67-92

Bussani, C., Cioni, R., Scarselli, B., Barciulli, F., Bucciantini, S., Simi, P., Fogli, A., Scarselli, C. (2002)

Strategies for the isolation and detection of foetal cells in transcervical samples. Prenat Diagn 12: 1098-101

Bulmer, J. N.,Cioni, R., Bussani, C., Cirigliano, V., Sole, F., Costa, C., Garcia, P., Adinolfi, M. (2003)

HLA-G positive trophoblastic cells in transcervical samples and their isolation and analysis by laser microdissection and QF-PCR. Prenat Diagn 2: 34-9

Mann, K., Fox, S. P., Abbs, S. J., Yau, S. C., Scriven, P. N., Docherty, Z., Ogilivie, C. M. (2001)

Development and implementation of a new rapid aneuploidy diagnostic service within the UK National Health Service and implications for the future of prenatal diagnosis. Lancet, Vol. 308

Adinolfi, M., Pertl, B., Sherlock, J. (1997)

Rapid detection of aneuploidies by microsatellite and the quantitative fluorescent polymerase chain reaction. Prenat. Diagn. 17(13): 1299-1311

Bili, C., Divane, A., Apesso, A., Konstantinos, T., Apostolos, A., Ionnis, B., Periklis, T., Florentin, L. (2002)

Prenatal diagnosis of common aneuploidies using quantitative fluorescent PCR. Prenat. Diagn. 22; 360-365

1. A method for detecting aneuploidy of a chromosome comprising:
a) obtaining a biological sample from a patient;
b) preparing DNA from said sample for subsequent analysis;
c) subjecting the DNA of said sample to a multiplex PCR reaction using a multiplicity of markers, at least two chromosomes of the DNA having a heterozygosity frequency sufficiently high to minimize the possibility that, if two alleles of that chromosome would normally be present in a normal diploid individual, those two alleles would both comprise the same variant of said STR;
d) comparing the results of said multiplex reaction with the results which might be expected from a normal diploid individual; and
e) thereby assessing the probability that said patient might be suffering from a chromosomal aneuploidy.

2. A method according to claim 1, wherein said markers comprise a combination of polymorphic markers for both sex chromosomes and autosomes.

3. A method according to claim 1, wherein one of said markers is a sequence derived from the Hypoxanthine Guanine Phosphoribosyltransferase gene, but modified by assigning new hybridization sites.

4. A method according to claim 3, wherein the modified marker comprises the primer shown in FIG. 1 and labeled “Mod.HPRT”.

5. A method according to claim 1, wherein at least two of said markers are selected from the primers shown in FIG. 1

6. A method according to claim 5, wherein the markers comprise the primer set shown in FIG. 1.

7. A method according to claim 1, wherein the DNA analysis is a PCR-based method.

8. A method according to claim 7, wherein the DNA analysis is by Quantitative Fluorescent PCR (QF-PCR).

9. A diagnostic kit for use in DNA analysis for detecting aneuploidy of a chromosome comprising:
a) highly conserved STR markers which have a heterozygosity frequency with respect to said chromosome which is sufficiently high to minimize the possibility that, if two alleles of that chromosome would normally be present in a diploid individual, those two alleles would both comprise the same variant of said STR marker; and
b) other standard reagents for use in multiplex PCR.

10. A diagnostic kit according to claim 9, wherein said markers comprise a combination of polymorphic markers for both sex chromosomes and autosomes.

11. A diagnostic kit according to claim 9, wherein one of said markers is a sequence derived from the Hypoxanthine Guanine Phosphoribosyltransferase gene, but modified by assigning new hybridisation sites.

12. A diagnostic kit according to claim 11, wherein the modified marker comprises the primer shown in FIG. 1 and referred to as “Mod.HPRT”.

13. A diagnostic kit according to claim 9, comprising at least two markers selected from the primers shown in FIG. 1.

14. A diagnostic kit according to claim 13, comprising the primer set shown in FIG. 1.

15. A diagnostic kit according to claim 9, wherein said multiplex PCR is a Quantitative Fluorescent PCR (QF-PCR).
